(19) United States
(12) Patent Application Publication
(10) Pub. No.: US 2012/0088827 A1
(43) Pub. Date: Apr. 12, 2012

(54) Oxabicyclo[4.1.0]Hept-B-en-S-yl Carbamoyl Derivatives Inhibiting The Nuclear Factor-Kappa (B) - (NF-KB)

(75) Inventors: Jie Zhang, Baltimore, MD (US); Drago Robert Sliskovic, Chelsea, MI (US); Charles E. Ducker, York, PA (US)

(73) Assignee: Profectus Biosciences, Inc., Baltimore, MD (US)

(21) Appl. No.: 13/378,385

(22) PCT Filed: Jun. 16, 2010
(86) PCT No.: PCT/US10/38744

§ 371 Date: Dec. 15, 2011

Related U.S. Application Data
(60) Provisional application No. 61/218,233, filed on Jun. 18, 2009.

(51) Int. Cl. A61K 31/336; A61P 35/00; A61P 29/00; A61P 9/10; A61P 3/10; A61P 31/00; A61P 9/00; C07D 303/46; A61P 37/00

(52) U.S. Cl. 514/475; 549/546

(57) ABSTRACT

The invention relates to compounds of formula (I), formula (II), formula (III) and formula (IV),

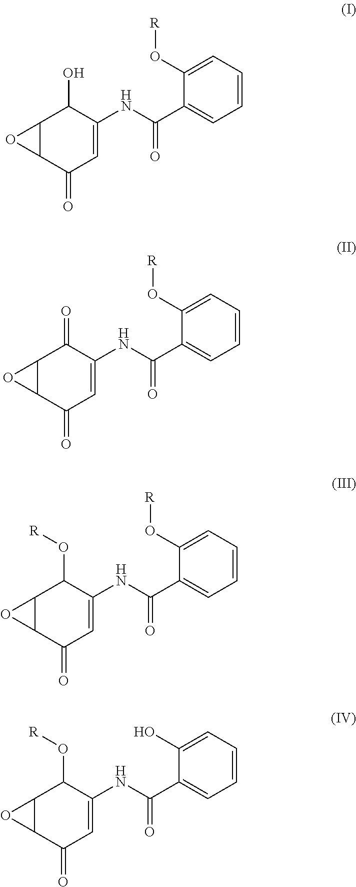

and pharmaceutically acceptable salts thereof for the treatment of cancer, inflammation, auto-immune diseases, diabetes and diabetic complications, infection, cardiovascular disease and ischemia-reperfusion injuries.

### FIELD OF INVENTION

The invention relates to compounds of formulas (I), (II), (III) and (IV)

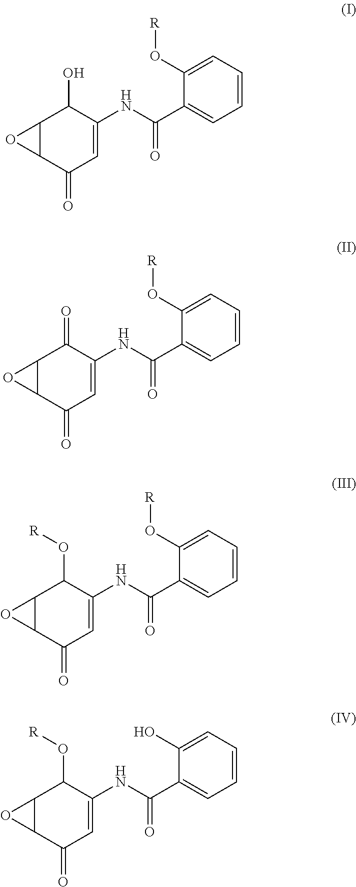

and pharmaceutically acceptable salts thereof for the treatment of cancer, inflammation, auto-immune disease, diabetes and diabetic complications, infection, cardiovascular disease and ischemia-reperfusion injuries.

### BACKGROUND OF INVENTION

Nuclear factor-kappaB (NF-κB) activation has been implicated in a wide variety of diseases, including cancer, diabetes mellitus, cardiovascular diseases, autoimmune diseases, viral replication, septic shock, neurodegenerative disorders, ataxia telangiectasia (AT), arthritis, asthma, inflammatory bowel disease, and other inflammatory conditions. For example, activation of NF-κB by the Gram-negative bacterial lipopolysaccharide (LPS) may contribute to the development of septic shock because NF-κB over-activates transcription of numerous cytokines and modifying enzymes, whose prolonged expression can negatively affect the function of vital organs such as the heart and liver (Arcaroli et al., 2006; Niu et al., 2008).

Similarly, autoimmune diseases such as systemic lupus erythematosus may also involve activation of NF-κB. The NF-κB transcription factor is critical for proper dendritic cell maturation, the loss of which is the hallmark of systemic lupus erythematosus (Kalergis et al., 2008; Kurylowicz & Nauman, 2008). Additionally, in chronic Alzheimer's disease, the amyloid β peptide causes production of reactive oxygen intermediates and indirectly activates gene expression through NF-κB sites (Giri et al., 2005).

Destructive erosion of bone or osteolysis is a major complication of inflammatory conditions such as rheumatoid arthritis (RA), periodontal disease, and periprosthetic osteolysis. RA is an autoimmune disease that affects approximately 1.0% of US adults, with a female to male ratio of 2.5 to 1 (Lawrence et al., 1998). Its hallmark is progressive joint destruction which causes major morbidity. Periodontal disease is highly prevalent and can affect up to 90% of the world's population. It is well known as the leading cause of tooth loss in adults (Pihlstrom et al., 2005). Despite its prevalence, little is known about the mechanism by which periodontal bone erosion occurs, although host response to pathogenic microorganisms present in the mouth appears to trigger the process. Periprosthetic osteolysis is caused by chronic bone resorption around exogenous implant devices until fixation is lost (Harris, 1995), and is considered as resulting from an innate immune response to wear-debris particles, with little contribution by components of the acquired immune system (Goldring et al., 1986).

Although these conditions are initiated by distinct causes and progress by alternative pathways, the important common factor(s) in the pathological process of these diseases are over-production of proinflammatory cytokines which is driven by constitutive activation of the NF-κB pathway in the inflamed tissue. The bone erosion seen in these conditions is largely localized to the inflamed tissues, distinct from systemic, hormonally regulated bone pathologies, such as osteoporosis. These inflamed tissues, found in many of these diseases, also produce proinflammatory cytokines, i.e., TNF-a, IL-1, and IL-6, that are, in turn, involved in osteoclast differentiation signaling and bone-resorbing activities. Thus, inflammatory osteolysis is the product of enhanced osteoclast recruitment and activation prompted by NF-κB driven proinflammatory cytokines in the inflamed tissue.

Inflammatory bowel disease (IBD) encompasses a number of chronic relapsing inflammatory disorders involving the gastrointestinal tract. The two most prevalent forms of IBD, Crohn's disease and ulcerative colitis, can be distinguished by unique histopathologies and immune responses (Atreya et al., 2008; Bouma & Strober, 2003). The limited efficacy and potential adverse effects of current treatments leave patients and doctors eager for new treatments to manage the chronic relapsing inflammatory nature of these diseases.

Although the exact aetiologies leading to Crohn's disease and ulcerative colitis remain unknown, they are generally thought to result from an inappropriate and ongoing activation of the mucosal immune system against the normal luminal flora (Tilg et al., 2008). As a result, resident macrophages, dendritic cells and T cells are activated and begin to secrete predominantly NF-κB-dependent chemokines and cytokines. NF-κB mediated overproduction of key pro-inflammatory mediators is attributed to the initiation and progression of both human IBD and animal models of colitis (Neurath et al., 1998; Wirtz & Neurath, 2007). In particular, macrophages of patients with IBD exhibit high levels of NF-κB DNA binding activity accompanied by increased production of interleukin (IL) 1, IL6 and tumor necrosis factor (TNF)α (Neurath et al., 1998). In addition, NF-κB plays a vital role in activating T helper cell 1 (Th1) and T helper cell 2 (Th2) cytokines, both of which are required for promoting and maintaining inflammation (Barnes, 1997). Because of the central role played by NF-κB in IBD, extensive efforts have been made to develop treatments targeting this pathway.

NF-κB has been shown to be constitutively expressed in numerous cancer derived cell lines from breast, ovarian, colon, pancreatic, thyroid, prostate, lung, head and neck, bladder, and skin tumors (Calzado et al., 2007). This has also been seen for B-cell lymphoma, Hodgkin's disease, T-cell lymphoma, adult T-cell leukemia, acute lymphoblastic leukemia, multiple myeloma, chronic lymphocytic leukemia, and acute myelogenous leukemia. NF-κB is a key mediator of normal inflammation as part of the defense response; however, chronic inflammation can lead to cancer, diabetes, and a host of other diseases as mentioned above. Several pro-inflammatory gene products have been identified that mediate a critical role in the carcinogenic process, angiogenesis, invasion, and metastasis of tumor cells. Among these gene products are TNF and members of its superfamily, IL-1alpha, IL-1beta, IL-6, IL-8, IL-18, chemokines, MMP-9, VEGF, COX-2, and 5-LOX. The expression of all these genes are mainly regulated by the transcription factor NF-κB, which is constitutively active in most tumors and is induced by carcinogens (such as cigarette smoke), tumor promoters, carcinogenic viral proteins (HIV-tat, KHSV, EBV-LMP1, HTLV1-tax, HPV, HCV, and HBV), chemotherapeutic agents, and gamma-irradiation (Aggarwal et al., 2006). These observations imply that anti-inflammatory agents that suppress NF-κB should have a potential in both the prevention and treatment of cancer.

The influenza virus protein hemagglutinin also activates NF-κB, and this activation may contribute to viral induction of cytokines and to some of the symptoms associated with influenza (Flory et al., 2000; Pahl & Baeuerle, 1995).

Oxidized lipids from the low density lipoproteins associated with atherosclerosis activate NF-κB, which then activates other genes such as inflammatory cytokines (Liao et al., 1994). Furthermore, mice that are susceptible to atherosclerosis exhibit NF-κB activation when fed an atherogenic diet due to their susceptibility to aortic atherosclerotic lesion formation associated with the accumulation of lipid peroxidation products, induction of inflammatory genes, and the activation of NF-κB transcription factors (Liao et al., 1994). Another important contributor to atherosclerosis is thrombin, which stimulates the proliferation of vascular smooth muscle cells through the activation of NF-κB (Maruyama et al., 1997). A truncated form of IκB repressor protein (IκBα) was shown to be the cause of the hypersensitive to ionizing radiation and are defective in the regulation of DNA synthesis in ataxia telangiectasia (AT) cells, which have constitutive levels of an NF-κB-activation (Jung et al., 1995). This mutation in the IκBα from the AT cells was shown to inactivate the repressor protein causing the constitutive activation of the NF-κB pathway. In light of all these findings, the abnormal activation or expression of NF-κB is clearly associated with a wide variety of pathologic conditions.

The infection and life-cycle of HIV-1 is tightly coupled to the NF-κB pathway in human mononuclear cells. Viral infection leads to the activation of NF-κB which generates the over stimulation and eventual depletion of T-cells that is the hallmark of AIDS (reviewed in (Argyropoulos & Mouzaki, 2006)). For instance, the expression of CCR5, a key receptor for HIV-1, is regulated by NF-κB (Liu et al., 1998). Deletion analysis of the CCR-5 promoter has demonstrated that loss of the 3′-distal NF-κB/AP-1 site drops transcription by >95% (Liu et al., 1998). These studies would suggest that constitutive repression of NF-κB would cause a dramatic decrease in CCR-5 receptor message. Since HIV-1 entry kinetics are influenced by expressed levels of CCR5 on the target T-cell surface (Ketas et al., 2007; Platt et al., 1998; Reeves et al., 2002), down modulating CCR5 may constrain the expansion of the pool of infected cells that spawns the viral reservoir. CXCR4 expression has also been reported to be influenced by NF-κB (Helbig et al., 2003) suggesting that NF-κB inhibitors may be equally effective against X4-tropic isolates that appear during late-stage infection. NF-κB is required for transcription of the integrated DNA-pro-virus (Baba, 2006; Iordanskiy et al., 2002; Mukerjee et al., 2006; Palmieri et al., 2004; Rizzi et al., 2004; Sui et al., 2006; Williams et al., 2007). In fact, lack of NF-κB activation leads to the generation of a population of cells harboring latent virus which is a major block to eliminating the virus from infected patients (Williams et al., 2006).

NF-κB promotes the expression of over 150 target genes in response to inflammatory stimulators. These genes include; interleukin-1, -2, -6 and the tumor necrosis factor receptor (TNF-R) (these receptor mediate apoptosis, and function as regulators of inflammation), as well as genes encoding immunoreceptors, cell adhesion molecules, and enzymes such as cyclooxygenase-II and inducible nitric oxide synthase (iNOS) (Karin, 2006; Tergaonkar, 2006). It also plays a key role in the progression of diseases associated with viral infections such as HCV and HIV-1.

Members of the NF-κB family include RelA/p65, RelB, c-Rel, p50/p105 (NF-κB1), and p52/p100 (NF-κB2) (Hayden & Ghosh, 2004; Hayden et al., 2006a; Hayden et al., 2006b). The Rel family members function as either homodimers or heterodimers with distinct specificity for cis-binding elements located within the promoter domains of NF-κB-regulated genes (Bosisio et al., 2006; Natoli et al., 2005; Saccani et al., 2004). Classical NF-κB, composed of the RelA/p65 and p50 heterodimer, is the best-studied form of NF-κB (Burstein & Duckett, 2003; Hayden & Ghosh, 2004) and references therein). Prior to cellular stimulation, classical NF-κB resides in the cytoplasm as an inactive complex bound to the IκBα inhibitor proteins. Inducers of NF-κB such as bacterial lipopolysaccharides, inflammatory cytokines, or HIV-1 Vpr protein release active NF-κB from the cytoplasmic complex by activating the IκB-kinase complex (IKK), which phosphorylates IκBα (Greten & Karin, 2004; Hacker & Karin, 2006; Israel, 2000; Karin, 1999; Scheidereit, 2006). Phosphorylation of IκB marks it for subsequent ubiquitinylation and degradation by the 26S proteosome. Free NF-κB dimers translocate into the nucleus where they stimulate the transcription of their target genes.

The molecular design of racemic dehydroxymethylepoxyquinomicin (DHMEQ) was based on the antibiotic epoxyquinomicin C isolated from Amycolatopsis (Chaicharoenpong et al. 2002). DHMEQ was synthesized as a racemate from 2,5-dimethoxyaniline in five steps. Separation of the enantiomers on a chiral column produced both (+) and (−) enantiomers. The (−)-enantiomer was shown to be more potent at inhibiting NF-κB than the (±)-enantiomer (Umezawa et al. 2004). DHMEQ has been characterized to specifically inhibit the translocation of NF-κB into the nucleus (Ariga et al. 2002). Specifically, it covalently modifies specific cysteine residues in p65 and other Rel homology proteins with a 1:1 stoichiometry ration (Yammamoto et al. 2008). As an NF-κB inhibitor, DHMEQ has been tested extensively in various animal models of diseases and demonstrated a broad spectrum of efficacy including treating solid tumors, hematological malignancy, arthritis, bowel ischemia, and atherosclerosis (Watanabe et al. 2006). Thus, DHMEQ may be useful as a novel treatment for cancer and inflammation (Takeuchi et al. 2003).

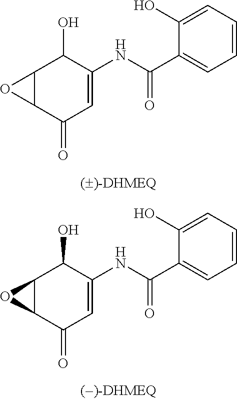

### SUMMARY OF THE INVENTION

The present invention relates to compounds having the structure of formulas (I), (II), (III) and (IV)

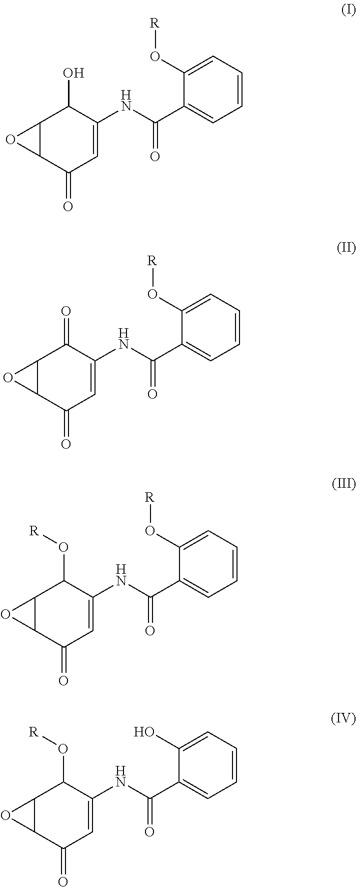

and pharmaceutically acceptable salts thereof, wherein each R is independently COR¹, CONHR¹, CONR¹R¹, COOR¹, CH₂OCOR¹, P(O)(OH)₂, P(O)(O(C1-C6)alkyl)₂, P(O)(O(C1-C6)alkylphenyl)₂, P(O)(OCH₂OCO(C1-C6)alkyl)₂, P(O)(OH)(OCH₂OCO(C1-C6)alkyl), P(O)(OH)(OC1-C6)alkyl), or P(O)(OH)(C1-C6)alkyl), P(O)(O(C1-C6)alkyl)₂, P(O)(OCH₂OCO(C1-C6)alkyl)₂, P(O)(OH)(OCH₂OCO(C1-C6)alkyl), P(O)(OH)(OC1-C6)alkyl), P(O)(OH)(C1-C6)alkyl), glycosyl (the radical resulting from the removal of a hydroxyl group of the hemiacetal form of a carbohydrate), or a salt thereof, wherein each R¹ is independently C1-C8 alkyl, trifluoromethyl, cycloalkyl, heterocycloalkyl, aryl, alkylaryl, heteroaryl or alkylheteroaryl, wherein the aryl or heteroaryl ring is substituted with 0 to 4 groups selected from fluorine, chlorine, bromine, cyano, hydroxyl, amino, trifluoromethyl, (C1-C4)alkyl, (C1-C4)alkoxy, pyridinyl, pyrimidinyl or benzyl optionally substituted with fluorine, chlorine, bromine, hydroxyl, trifluoromethyl, (C1-C4)alkyl or (C1-C4) alkoxy.

The present invention also relates to a pharmaceutical composition comprising a compound of any one formula (I), formula (II), formula (III), or formula (IV) or a pharmaceutically acceptable salt thereof, and a pharmaceutically acceptable carrier.

The present invention also relates to particular compounds of formula (I), formula (II), formula (III) and formula (IV) having the structures of formula (V), formula (VI), formula (VII) and formula (VIII), respectively,

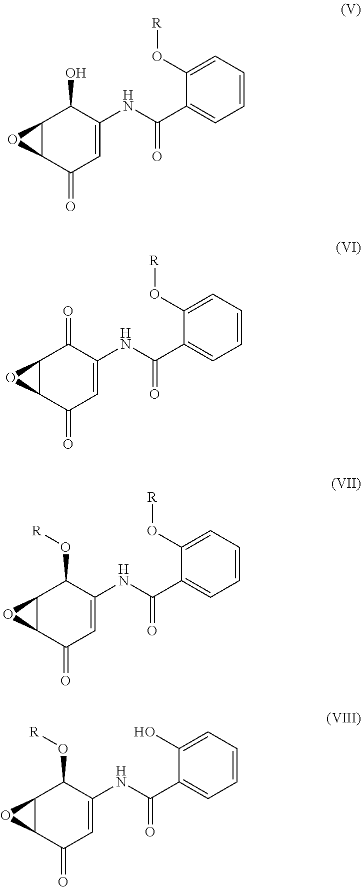

wherein R is defined above for formulas (I), (II), (III) and (IV), and pharmaceutically acceptable salts thereof.

The present invention further relates to a method of treating cancer, inflammation, auto-immune disease, diabetes and diabetic complications, infection, cardiovascular disease and ischemia-reperfusion injuries, comprising administering to a mammal in need of such treatment, such as a human, a therapeutically effective amount of a compound of any one of formulas (I)-(VIII), or a pharmaceutically acceptable salt thereof.

The present invention additionally relates to a method of inhibiting gene expression and signal transduction directly or indirectly through the NF-κB pathway in a mammal, such as a human, comprising administering to a mammal in need of such a treatment a therapeutically effective amount of a compound of any one of formulas (I) to (VIII), or a pharmaceutically acceptable salt thereof.

### DETAILED DESCRIPTION

### Definitions

The terms used to describe the present invention have the following meanings herein. The compounds and intermediates of the present invention may be named according to either the IUPAC (International Union for Pure and Applied Chemistry) or CAS (Chemical Abstracts Service) nomenclature systems.

The carbon atom content of the various hydrocarbon-containing moieties herein may be indicated by a prefix designating the minimum and maximum number of carbon atoms in the moiety, for example, the prefix (Ca-Cb)alkyl indicate an alkyl moiety of the integer “a” to “b” carbon atoms, inclusive. Thus, for example, (C1-C6) alkyl refers to an alkyl group of one to six carbon atoms inclusive. The term “alkyl” denotes a straight or branched chain of carbon atoms with only hydrogen atom substituents, wherein the carbon chain optionally contains one or more double or triple bonds, or a combination of double bonds and triple bonds. Examples of alkyl groups include, but are not limited to, methyl, ethyl, propyl, isopropyl, propenyl, propynyl, hexadienyl, and the like.

The term “alkoxy” refers to straight or branched, monovalent, saturated aliphatic chains of carbon atoms wherein one of the carbon atoms has been replaced with an oxygen atom. Examples of alkoxy groups include, but are not limited to, methoxy, ethoxy and iso-propoxy.

The term “cycloalkyl” refers to saturated and unsaturated nonaromatic monocyclic or bicyclic ring systems containing only carbon atoms as ring atoms. Examples of cycloalkyl groups include, but are not limited to, cyclopropyl, cyclobutyl, cyclopentyl and cyclohexenyl. Cycloalkyl groups may also be optionally fused to aryl rings such as, for example, but not limited to, benzene to form fused cycloalkyl groups, such as indanyl and the like.

The term “heteroalkyl” refers to saturated and unsaturated nonaromatic monocyclic or bicyclic ring systems containing from 1 to 4 heteroatoms as ring atoms. Examples of heteroalkyl groups include, but are not limited to, pyrrolidinyl, piperidinyl, piperazinyl, tetrahydrofuranyl, dioxanyl and morpholinyl. Heteroalkyl groups may also be optionally fused to aryl rings such as, for example, but not limited to benzene to form fused heteroalkyl groups, such as dihydroindolyl and the like.

The term “heteroatom” refers to nitrogen, oxygen and sulfur atoms.

The term “aryl” refers to aromatic monocyclic and bicyclic rings systems containing only carbon atoms as ring atoms. Examples include, but are not limited to, phenyl and naphthyl.

The term “heteroaryl” refers to aromatic monocyclic and bicyclic ring systems containing from 1 to 5 heteroatoms as ring atoms. Examples include pyrrolyl, furanyl, thienyl, imidazolyl oxazolyl, isoxazolyl, thiazolyl, isothiazolyl, pyrazolyl, 1,2,3-triazolyl, 1,2,5-thiadiazolyl, 1,2,3-thiadiazolyl, 1,2,3-oxadiazolyl, 1,2,5-oxadiazolyl, pyridinyl, pyrimidinyl, pyrazinyl, pyridazinyl, triazinyl, benzofuranyl, isobenzofuranyl, benzothienyl, isobenzothienyl, indolizinyl, indolyl, isoindolyl, benzoxazolyl, benzimidazolyl, indazolyl, benzisoxazolyl, benzisothiazolyl, benzopyrazolyl, benzoxadiazolyl, benzothiadiazolyl, benzotriazolyl, quinolinyl, isoquinolinyl, cinnolinyl, quinolizinyl, phthalazinyl, quinoxalinyl, quinazolinyl, naphthyridinyl, pteridinyl, pyrrolopyridinyl, thienopyridinyl, furanopyridinyl, isothiazolopyridinyl, thiazolopyridinyl, isoxazolopyridinyl, oxazolopyridinyl, pyrazolopyridinyl, imidazopyridinyl, pyrrolopyrazinyl, thienopyrazinyl, furanopyrazinyl, isothiazolopyrazinyl, thiazolopyrazinyl, isoxazolopyrazinyl, oxazolopyrazinyl, pyrazolopyrazinyl, imidazopyrazinyl, pyrrolopyrimidinyl, thienopyrimidinyl, furanopyrimidinyl, isothiazolopyrimidinyl, thiazolopyrimidinyl, isoxazolopyrimidinyl, oxazolopyrimidinyl, pyrazolopyrimidinyl, imidazopyrimidinyl, pyrrolopyridazinyl, thienopyridazinyl, furanopyridazinyl, isothiazolopyridazinyl, thiazolopyridazinyl, isoxazolopyridazinyl, oxazolopyridazinyl, pyrazolopyridazinyl, imidazopyridazinyl, oxadiazolopyridinyl, thiadiazolopyridinyl, triazolopyridinyl, oxadiazolopyrazinyl, thiadiazolopyrazinyl, triazolopyrazinyl, oxadiazolopyrimidinyl, thiadiazolopyrimidinyl, triazolopyrimidinyl, oxadiazolopyridazinyl, thiadiazolopyridazinyl, triazolopyridazinyl, isoxazolotriazinyl, isothiazolotriazinyl, pyrazolotriazinyl, oxazolotriazinyl, thiazolotriazinyl, imidazotriazinyl, oxadiazolotriazinyl, thiadiazolotriazinyl, triazolotriazinyl, carbazolyl and the like.

The term “alkylaryl” refers to an alkyl group substituted by an aryl group.

The term “alkylheteroaryl” refers to an alkyl group substituted by a heteroaryl group.

The term “halo” refers to chloro, bromo, fluoro, or iodo.

The term “substituted” refers to a hydrogen atom on a molecule that has been replaced with a different atom or molecule. The atom or molecule replacing the hydrogen atom is denoted as a “substituent.”

The phrase “therapeutically effective amount” refers to an amount of a compound that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition.

The phrase “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “mammal” relates to an individual animal that is a member of the taxonomic class Mammalia. Examples of mammals include, but are not limited to, humans, dogs, cats, horses and cattle. In the present invention, the preferred mammal is a human.

In an exemplary embodiment, the compounds of the present invention have the structure shown in any one of formula (V), formula (VI), formula (VII) and formula (VIII).

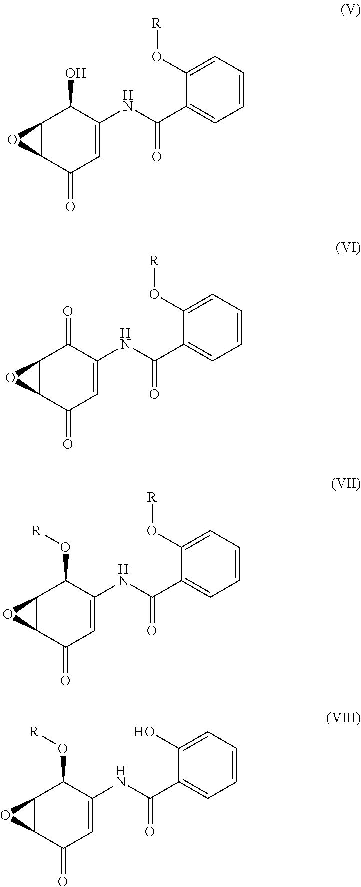

The compounds of the invention may be resolved into their pure enantiomers by methods known to those skilled in the art, for example by formation of diastereoisomeric salts which may be separated, for example, by crystallization; formation of diastereoisomeric derivatives or complexes which may be separated (for example, by crystallization, gas-liquid or liquid chromatography); selective reaction of one enantiomer with an enantiomer-specific reagent (for example, enzymatic esterification); or gas-liquid or liquid chromatography in a chiral environment, for example, on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, the specific stereoisomers may be synthesized by using an optically active starting material, by asymmetric synthesis using optically active reagents, substrates, catalysts or solvents, or by converting one stereoisomer into the other by asymmetric transformation.

Wherein the compounds contain one or more additional stereogenic centers, those skilled in the art will appreciate that all diastereoisomers and diastereoisomeric mixtures of the compounds illustrated and discussed herein are within the scope of the present invention. These diastereoisomers may be isolated by methods known to those skilled in the art, for example, by crystallization, gas-liquid or liquid chromatography. Alternatively, intermediates in the course of the synthesis may exist as racemic mixtures and be subjected to resolution by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example, enzymatic esterification; or gas-liquid or liquid chromatography in a chiral environment, for example, on a chiral support for example silica with a bound chiral ligand or in the presence of a chiral solvent. It will be appreciated that where the desired stereoisomer is converted into another chemical entity by one of the separation procedures described above, a further step is required to liberate the desired enantiomeric form. Alternatively, the specific stereoisomers may be synthesized by using an optically active starting material, by asymmetric synthesis using optically active reagents, substrates, catalysts or solvents, or by converting one stereoisomer into the other by asymmetric transformation. These methods are described in more detail in texts such as “Chiral Drugs”, Cynthia A. Challener (Editor), Wiley, 2002 or “Chiral Drug Separation” by Bingyunh Li and Donald T. Haynia in “Encyclopedia of Chemical Processing” by Sunggyu Lee and Lee Lee (Editors), CRC Press, 2005.

The compounds of the present invention, and the salts thereof, may exist in the unsolvated as well as the solvated forms with pharmaceutically acceptable solvents such as water, ethanol, and the like.

Selected compounds of formulas (I)-(VIII) and their salts and solvates may exist in more than one crystal form. Polymorphs of compounds represented by formulas (I)-(VIII) form part of this invention and may be prepared by crystallization of a compound of formulas (I)-(VIII) under different conditions. For example, using different solvents or solvent mixtures for recrystallization; crystallization at different temperatures; various modes of cooling, ranging from very fast to very slow cooling during crystallization. Polymorphs may also be obtained by heating or melting a compound of formulas (I)-(VIII) followed by gradual or fast cooling. The presence of polymorphs may be determined by solid state NMR spectroscopy, IR spectroscopy, differential scanning calorimetry, powder X-ray diffraction or other such techniques.

This invention also includes isotopically-labeled compounds, which are identical to those described by formulas (I)-(VIII), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, sulfur and fluorine, such as ²H, ³H, ¹³C, ¹⁴C, ¹⁵N, ¹⁸O, ¹⁷O, ³⁵S, ³⁶Cl, ¹²⁵I, ¹²⁹I and ¹⁸F respectively. Compounds of the present invention and pharmaceutically acceptable salts of the compounds which contain the aforementioned isotopes and/or other isotopes of other atoms are within the scope of this invention. Certain isotopically-labeled compounds of the present invention, for example those into which an isotope such as ²H(deuterium) are incorporated can afford certain therapeutic advantage resulting from greater metabolic stability, for example, increased in vivo half life or reduced dosage requirements and, hence, may be preferred in some circumstances. Isotopically labeled compounds of formulas (I)-(VIII) of this invention, salts and solvates thereof can generally be prepared by carrying out procedures disclosed in the schemes and/or in the Examples below, by substituting a readily available isotopically labeled reagent for a non-isotopically labeled reagent.

Pharmaceutically acceptable salts, as used herein in relation to compounds of the present invention, include pharmaceutically acceptable inorganic and organic salts of said compounds. These salts can be prepared in situ during the final isolation and purification of a compound, or by separately reacting the compound with a suitable organic or inorganic acid and isolating the salt thus formed. Representative salts include, but are not limited to, the hydrobromide, hydrochloride, hydroiodide, sulfate, bisulfate, nitrate, acetate, trifluoroacetate, oxalate, besylate, camsylate, palmitate, malonate, stearate, laurate, malate, borate, benzoate, lactate, phosphate, hexafluorophosphate, benzene sulfonate, tosylate, formate, citrate, maleate, fumarate, succinate, tartrate, naphthylate, mesylate, glucoheptonate, lactobionate, and laurylsulphonate salts, and the like. Compounds of the present invention may also react to form salts with pharmaceutically acceptable metal and amine cations formed from organic and inorganic bases. The term “pharmaceutically acceptable metal cation” contemplates positively charged metal ions derived from sodium, potassium, calcium, magnesium, aluminum, iron, zinc and the like. The term “pharmaceutically acceptable amine cation” contemplates the positively charged ions derived from ammonia and organic nitrogenous bases strong enough to form such cations. Bases useful for the formation of pharmaceutically acceptable nontoxic base addition salts of compounds of the present invention form a class whose limits are readily understood by those skilled in the art. (See, for example, Berge, et “Pharmaceutical Salts,” J. Pharm. Sci., 66:1-19 (1977)).

The term “prodrug” is intended to refer to a compound that is transformed in vivo to yield a compound of formula (I) or a pharmaceutically acceptable salt or solvate of the compound. This transformation may occur by various mechanisms, such as, for example, through hydrolysis in blood. A prodrug of a compound of formulas (I)-(VIII) may be formed, for example, in a conventional manner from functional groups such as with an amino, hydroxy or carboxy. A discussion of the use of prodrugs is provided by T. Higuchi and W. Stella, “Pro-drugs as Novel Delivery Systems,” Vol. 14 of the A.C.S. Symposium Series, and in “Bioreversible Carriers in Drug Design”, ed. Edward B. Roche, American Pharmaceutical Association and Pergamon Press, 1987. In an aspect of the invention, compounds (I) to (VIII) are intended to serve as prodrugs for DHMEQ. However, because each of these compounds also contains a “NH” moiety which may be further derivatized, the invention also includes prodrugs of the compounds of formulas (I) to (VIII) resulting from such derivatization. In addition, compounds (I), (IV), (V) and (VIII) contain a hydroxy (OH) moiety which may also be derivatized to create additional prodrugs.

In general, compounds of the present invention may be prepared by the general synthetic methods outlined in reaction Schemes 1-5. These methods are simply illustrative of particular embodiments and are not intended to further limit the invention.

Compound 1 was prepared according to the method of Umezawa (Suzuki, Y.; Sugiyama, C.; Ohno, O.; Umezawa, K.: Tetrahedron (2004), 60, 7061-7066. The reaction of compound (1) with an acid chloride (R¹COCl) in a solvent such as, for example, but not limited to, acetone or tetrahydrofuran with a base such as, for example, but not limited to, potassium carbonate or pyridine gives the esters (2) or (3). The production of either compound (2) or (3) is dependent upon the stoichiometry of the acid chloride employed: one equivalent produces the mono-ester (2) while two equivalents produce the bis-esters (3) as shown in Scheme 1. The reaction of compound (1) with an acid chloride (R¹COCl) in a solvent such as, but not limited to tetrahydrofuran with a base such as, for example, but not limited to, sodium hydride gives the ester (4). The reaction of compound (1) with chloroformates (R¹OCOCl) in a solvent such as, for example, but not limited to, tetrahydrofuran with a base such as, for example, but not limited to, pyridine gives the carbonates (5) or (6). The production of either compound (5) or (6) is dependent upon the stoichiometry of the chloroformate employed: one equivalent produces the mono-carbonate (5) while two equivalents the bis-carbonates (6) as shown in Scheme 2. The reaction of compound (1) with a chloroformate (R¹OCOCl) in a solvent such as, for example, but not limited to, tetrahydrofuran with a base such as, for example, but not limited to, potassium carbonate gives the carbonate (7). The reaction of compound (1) with isocyanates (R¹NCO) in a solvent such as, for example, but not limited to, dichloromethane with a catalytic amount of a base such as, for example, but not limited to, triethylamine gives the carbamates (8) or (9). The production of either compound (8) or (9) is dependent upon the stoichiometry of the isocyanate employed: one equivalent produces the mono-carbamate (8) while two equivalents produce the bis-carbamates (9) as shown in Scheme 3. The reaction of compound (1) with an isocyanate (R¹NCO) in a solvent such as, for example, but not limited to, tetrahydrofuran gives the carbamate (10). The reaction of compound (1) with a phosphorylating agent such as, for example, but not limited to, ClP(O)(OCH₃)₂ in a solvent such as, for example, but not limited to, tetrahydrofuran with a base such as, for example, but not limited to, triethylamine gives the phosphate ester (11) which can be further hydrolyzed to (12) using, for example, but not limited to, TMS-Br in a solvent such as, for example, but not limited to, dichloromethane as shown in Scheme 4. The reaction of compound (1) with, for example, but not limited to, an alkylcarbonyloxymethyl iodide R¹C(O)OCH₂I, (generated from the corresponding chloride, R¹C(O)OCH₂Cl in a modified Finkelstein reaction using sodium iodide in a mixed solvent of acetonitrile and dimethylformamide), in the presence of, for example, but not limited to, 1,8-bis(dimethylamino)naphthalene in, for example, but not limited to, dry acetonitrile gave compound (13). The use of two equivalents of alkylcarbonyloxymethyl iodide R¹C(O)OCH₂I gave the compounds (14). The reaction of compound (1) with, for example, but not limited to, an alkylcarbonyloxymethyl iodide R¹C(O)OCH₂I in a solvent such as, for example, but not limited to, tetrahydrofuran with a base such as, for example, but not limited to, sodium hydride gives compound (15) as shown in Scheme 5.

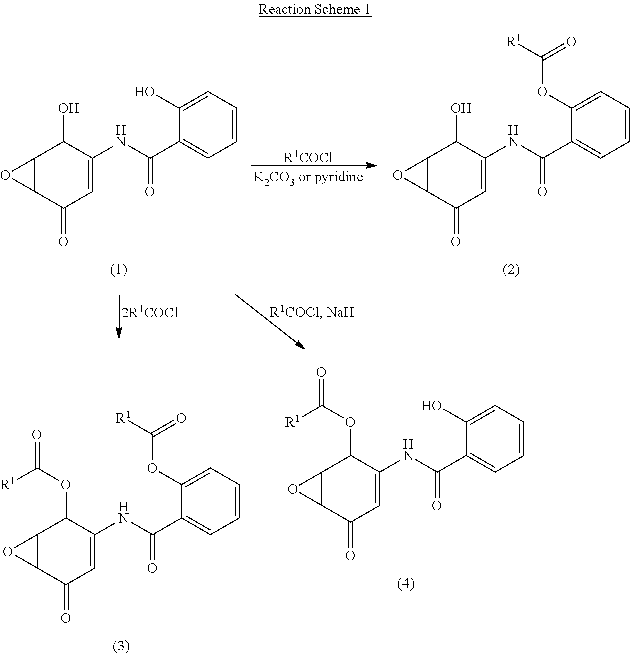

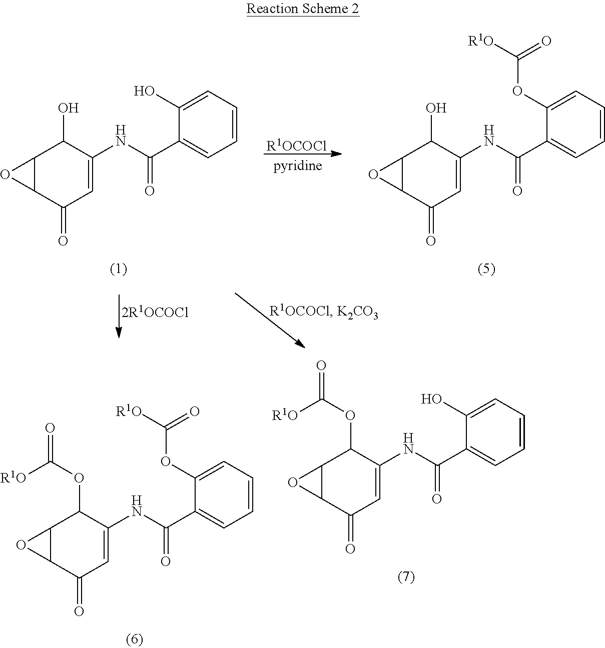

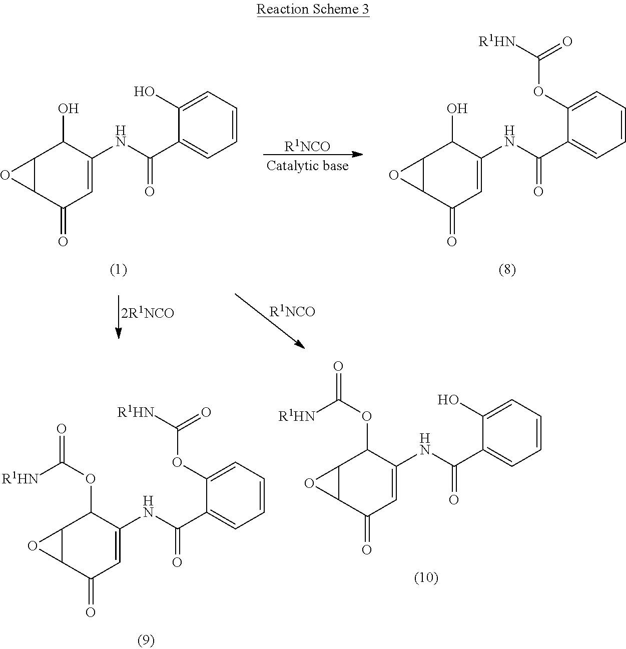

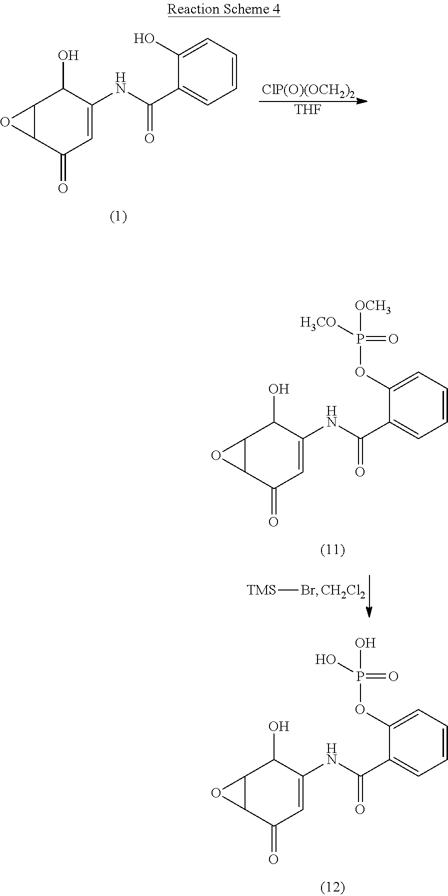

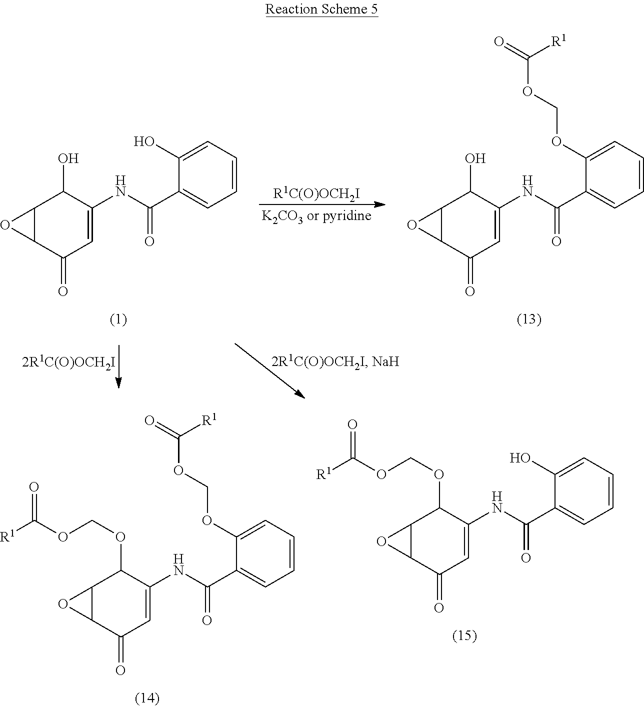

A pharmaceutical composition of the present invention comprises a therapeutically effective amount of a compound of formulas (I) to (IV), or a pharmaceutically acceptable salt thereof, and a pharmaceutically acceptable carrier, vehicle, diluent or excipient. An exemplary embodiment of a pharmaceutical composition of the present invention comprises a therapeutically effective amount of a compound of formulas (V) to (VIII), or a pharmaceutically acceptable salt thereof, and a pharmaceutically acceptable carrier, vehicle, diluent or excipient. The pharmaceutical compositions formed by combining the compounds of this invention and the pharmaceutically acceptable carriers, vehicles or diluents are then readily administered in a variety of dosage forms such as tablets, powders, lozenges, syrups, injectable solutions and the like. These pharmaceutical compositions can, if desired, contain additional ingredients such as flavorings, binders, excipients and the like.

Thus, for purposes of oral administration, tablets containing various excipients such as sodium citrate, calcium carbonate and/or calcium phosphate, may be employed along with various disintegrants such as starch, alginic acid and/or certain complex silicates, together with binding agents such as polyvinylpyrrolidone, sucrose, gelatin and/or acacia. Additionally, lubricating agents such as magnesium stearate, sodium lauryl sulfate and talc are often useful for tabletting purposes. Solid compositions of a similar type may also be employed as fillers in soft and hard filled gelatin capsules. Preferred materials for this include lactose or milk sugar and high molecular weight polyethylene glycols. When aqueous suspensions of elixirs are desired for oral administration, the active pharmaceutical agent therein may be combined with various sweetening of flavoring agents, coloring matter or dyes and, if desired, emulsifying or suspending agents, together with diluents such as water, ethanol, propylene glycol, glycerin and/or combinations thereof.

For parenteral administration, solutions of the compounds or compositions of this invention in sesame or peanut oil, aqueous propylene glycol, or in sterile aqueous solutions may be employed. Such aqueous solutions should be suitably buffered if necessary and the liquid diluents first rendered isotonic with sufficient saline or glucose. These particular aqueous solutions are especially suitable for intravenous, intramuscular, subcutaneous and intraperitoneal administration. In this connection, the sterile aqueous media employed are all readily available by standard techniques known to those skilled in the art.

In an exemplary embodiment, the pharmaceutical preparation is in unit dosage form. In such form, the preparation is subdivided into unit doses containing appropriate quantities of the active component. The unit dosage form can be a packaged preparation, for example, packeted tablets, capsules, and powders in vial or ampoules. The unit dosage form can also be a capsule, cachet, or tablet itself or it can be the appropriate number of any of these packaged forms.

Methods of preparing various pharmaceutical compositions with a certain amount of active ingredient are known to those skilled in the art. For examples of methods of preparing pharmaceutical compositions, see Remington: The Science and Practice of Pharmacy, Lippincott, Williams & Wilkins, 21^{st} ed. (2005), which is incorporated by reference in its entirety.

### EXAMPLES

### Example 1

### (±)-2-(2-hydroxy-5-oxo-7-oxabicyclo[4.1.0]hept-3-en-3-ylcarbamoyl)phenyl 3-methylbutanoate

Compound 1 was prepared according to the method of Umezawa (Suzuki, Y.; Sugiyama, C.; Ohno, O.; Umezawa, K.: Tetrahedron (2004), 60, 7061-7066. The ¹HNMR spectrum was consistent with that reported in the Umezawa reference.

In a 20 gram vial, compound 1 (100 mg, 0.383 mmol) and potassium carbonate (117 mg, 0.843 mmol) were suspended in acetone (5 mL). The reaction mixture was cooled to 0° C. and then iso-valeryl chloride (0.050 mL, 0.421 mmol) was added. The reaction mixture was stirred at 0° C. for 1 hour and then at 5-10° C. for 1 hour. The mixture was filtered and then concentrated and the residue was purified via silica gel chromatography (40% ethyl acetate in heptanes). The desired product was isolated as a white solid (39 mg, 30%). The product structure was confirmed by ¹HNMR (CDCl₃): δ 8.90 (s, IH), 7.85 (m, 1H), 7.55 (m, IH), 7.45 (m, 1H), 7.10 (m, 1H), 7.00 (s, 1H), 4.80 (m, 1H), 3.80 (m, 1H), 3.45 (m, 1H), 3.05 (m, 1H), 2.60 (m, 2H), 2.20 (m, 1H), 1.05 (m, 6H) ppm.

### Example 2

### (±)-3-(2-isopropoxycarbonyloxy-benzoylamino)-5-oxo-7-oxa-bicyclo[4.1.0]hept-3-en-2-yl ester isopropyl ester

In a 20 gram vial, compound (1) (100 mg, 0.383 mmol) and potassium carbonate (127 mg, 0.919 mmol) were suspended in acetone (4 mL). The reaction mixture was cooled to 0° C. and then iso-propyl chloroformate (0.84 mL, 0.843 mmol) was added. The reaction was stirred at 0° C. for 30 minutes and then at room temperature for 1 hour. The mixture was filtered, concentrated and the residue was purified via silica gel chromatography (15-40% ethyl acetate in heptanes). The fractions were allowed to sit at room temperature for 48 hours. The resulting crystals were filtered to yield the desired product (19 mg, 11%). The product structure was confirmed by ¹HNMR (CDCl₃): δ 8.10 (m, 1H), 7.65 (m, IH), 7.40 (m, 1H), 7.20 (m, 1H), 6.80 (s, 1H), 6.00 (m, 1H), 4.85 (m, 1H), 3.95 (m, 1H), 3.50 (m, 1H), 1.40 (m, 6H), 1.20 (m, 6H) ppm.

### Example 3

### (±)-2-(2-hydroxy-5-oxo-7-oxabicyclo[4.1.0]hept-3-en-3-ylcarbamoyl)phenyl 2-cyclohexylacetate

In a 20 gram vial, compound 1 (78 mg, 0.299 mmol) and potassium carbonate (62 mg, 0.448 mmol) were suspended in acetone (5 mL). The reaction was cooled to 0° C. and then cyclohexyl acetyl chloride (0.057 mL, 0.359 mmol) was added. The reaction was stirred at 0° C. for 30 minutes and then at room temperature for 6 hours. The mixture was concentrated and the residue was purified via silica gel chromatography (2% ethyl acetate in heptanes to 10% ethyl acetate in heptanes). The fractions containing the product were concentrated and then stored in the refrigerator in ethyl acetate/heptanes (1:2) for 72 h. The crystals were filtered and dried to yield the desired product as a white solid (29 mg, 25%). The product structure was confirmed by ¹HNMR (CDCl₃): δ 8.90 (s, IH), 7.85 (m, 1H), 7.55 (m, IH), 7.40 (m, 1H), 7.10 (m, 1H), 7.00 (s, 1H), 4.60 (m, 1H), 3.85 (m, 1H), 3.55 (m, 1H), 2.95 (m, 1H), 2.55 (m, 2H), 1.95 (m, 1H), 1.80 (m, 5H), 1.30 (m, 5H) ppm.

### Example 4

### (±)-2-(2-hydroxy-5-oxo-7-oxabicyclo[4.1.0]hept-3-en-3-ylcarbamoyl)phenyl 2-methylpentanoate

In a 20 gram vial, compound 1 (325 mg, 1.25 mmol) was suspended in tetrahydrofuran (12 mL) To this mixture, pyridine (0.11 mL, 1.37 mmol) and 2-methyl valeryl chloride (0.19 mL, 1.37 mmol) were added. The reaction was complete within 1 h. The reaction was filtered over a small pad of silica gel. The pad was washed with heptanes:ethyl acetate (1:1) and the eluant was concentrated. The crude solid was loaded onto a silica gel column. The final compound was isolated in three separate fractions (210 mg, 47% yield, >90% pure). The product structure was confirmed by ¹HNMR (CDCl₃): δ 8.70 (s, IH), 7.85 (1H), 7.55 (m, IH), 7.40 (m, 1H), 7.10 (m, 1H), 7.00 (s, 1H), 4.70 (m, 1H), 3.90 (m, 1H), 3.55 (m, 1H), 3.05 (m, 1H), 2.80 (m, 1H), 1.80 (m, 1H), 1.40-1.60 (m, 4H), 1.25 (m, 3H), 0.90 (m, 3H) ppm.

Employing the general methods previously described, the following compounds were prepared:

### Example 5

### (±)-2-(2-hydroxy-5-oxo-7-oxabicyclo[4.1.0]hept-3-en-3-ylcarbamoyl)phenyl 2-ethylhexanoate

The product structure was confirmed by ¹HNMR (CDCl₃): δ 8.75 (s, IH), 7.80 (m, 1H), 7.60 (m, IH), 7.40 (m, 1H), 7.10 (m, 1H), 7.00 (s, 1H), 4.70 (m, 1H), 3.90 (m, 1H), 3.55 (m, 1H), 3.10 (m, 1H), 2.60 (m, 1H), 1.80 (m, 1H), 1.75-1.00 (m, 11H), 0.90 (m, 3H) ppm.

### Example 6

### (±)-2-(2-hydroxy-5-oxo-7-oxabicyclo[4.1.0]hept-3-en-3-ylcarbamoyl)phenyl 3,3-dimethylbutanoate

The product structure was confirmed by ¹HNMR (acetone-d6): δ7.90 (m, 1H), 7.60 (m, IH), 7.45 (m, 1H), 7.25 (m, 1H), 6.95 (s, 1H), 4.95 (m, 2H), 3.95 (m, 1H), 3.40 (m, 1H), 2.60 (m, 2H), 1.05 (m, 9H) ppm.

Employing the general methods previously described, the following compounds were prepared:

### Example 7

### (±)-2-(2-hydroxy-5-oxo-7-oxabicyclo[4.1.0]hept-3-en-3-ylcarbamoyl)phenyl isopropyl carbonate (5a)

The product structure was confirmed by ¹HNMR (CDCl₃): δ 9.30 (s, IH), 7.90 (m, 1H), 7.60 (m, IH), 7.45 (m, 1H), 7.35 (m, 1H), 6.95 (s, 1H), 5.65 (m, 1H), 4.95 (m, 2H), 3.95 (m, 1H), 3.40 (m, 1H), 1.40 (m, 6H) ppm.

### Example 8

### (±)-1-hydroxy-N-(2-hydroxy-5-oxo-7-oxabicyclo[4.1.0]hept-3-en-3-yl)-2-naphthamide

In a 20-gram vial, compound 1 (200 mg, 0.77 mmol) was stirred in acetone (12 mL). To this solution was added potassium carbonate (266 mg, 1.92 mmol) and isopropyl chloroformate (0.54 mL, 0.54 mmol, 1.0M solution). The reaction appeared to be complete by LC/MS after 30 minutes. The crude mixture was filtered over a silica gel plug and washed with 50:50 ethyl acetate: heptanes. The solvent was evaporated by rotary evaporation to yield pure product (150 mg, 80%, >90% purity). The product structure was confirmed by ¹HNMR (CDCl₃): δ10.30 (br.s, 1H), 7.95 (m, 1H), 7.60 (m, 1H), 7.10 (m, 1H), 6.95 (m, 1H), 6.80 (m, 1H), 6.10 (m, 1H), 5.05 (m, 1H), 4.10 (m, 1H), 3.65 (m, 1H), 1.30 (m, 6H) ppm.

### Example 9

### (±)-2-(2-(3,3-dimethylbutanoyloxy)-5-oxo-7-oxabicyclo[4.1.0]hept-3-en-3-ylcarbamoyl)phenyl 3,3-dimethylbutanoate

In a 25-mL round bottom flask, compound 1 (100 mg, 0.38 mmol) was suspended in tetrahydrofuran (10 mL) and cooled to −78° C. To this mixture, lithium tert-butoxide (0.40 mL, 0.40 mmol, 1.0 M in tetrahydrofuran) was added. After 30 minutes, tert-butyl acetyl chloride (49 mg, 0.363 mmol) was added. The solution was diluted with ethyl acetate and washed with saturated aqueous ammonium chloride. The organic layer was washed with brine, dried over anhydrous sodium sulfate, filtered, and concentrated. The crude material was purified by column chromatography (eluting with pentanes: diethyl ether). The bis-ester was obtained (22 mg, 12.5%, >90% pure) and the product structure was confirmed by ¹HNMR (d₆-acetone): δ9.20 (br.s, 1H), 7.75 (m, 1H), 7.60 (m, 1H), 7.40 (m, 1H), 7.10 (m, 1H), 6.95 (m, 1H), 6.10 (m, 1H), 4.10 (m, 1H), 3.55 (m, 1H), 2.50 (m, 4H), 1.10 (m, 18H) ppm.

### Example 10

### (±)-diethyl 2-(2-hydroxy-5-oxo-7-oxabicyclo[4.1.0]hept-3-en-3-ylcarbamoyl)phenyl phosphate

In a 20-gram vial, compound 1 (200 mg, 0.766 mmol) was stirred with tetrahydrofuran (8 mL). Triethylamine (0.53 mL, 3.83 mmol) and diethyl chlorophosphate (0.105 mL, 0.728 mmol) were added. The reaction was complete after 10 minutes as determined by LC/MS. The crude mixture was filtered and then concentrated in vacuo. The crude oil was purified by column chromatography (eluting with heptanes: ethyl acetate). The phospho-ester was obtained (160 mg, 53%, >90% pure) and the product structure was confirmed by ¹HNMR (d₆-acetone): δ9.40 (br.s, 1H), 7.90 (m, 1H), 7.60 (m, 1H), 7.50 (m, 1H), 7.40 (m, 1H), 6.95 (m, 1H), 4.90 (m, 1H), 4.25 (m, 4H), 3.90 (m, 1H), 3.40 (m, 1H), 1.30 (m, 6H) ppm.

### Example 11

### (±)-3-(2-hydroxybenzamido)-5-oxo-7-oxabicyclo[4.1.0]hept-3-en-2-yl phenylcarbamate

In a 20-gram vial, compound 1 (250 mg, 0.958 mmol) was stirred with tetrahydrofuran (10 mL). To this mixture, phenyl isocyanate (0.10 mL, 0.956 mmol) was added and the solution was stirred at room temperature overnight. The reaction mixture was filtered, concentrated in vacuo, and purified by column chromatography (eluting with heptanes: ethyl acetate). The carbamate was isolated (70 mg, 19%, >97% pure) and the product structure was confirmed by ¹HNMR (d₆-acetone): δ9.20 (br.s, 1H), 7.90 (m, 1H), 7.60 (m, 2H), 7.40 (m, 3H), 7.10 (m, 2H), 6.95 (m, 2H), 6.10 (m, 1H), 4.10 (m, 1H), 3.50 (m, 1H) ppm.

### Example 12

### (±)-dibenzyl 2-(2-hydroxy-5-oxo-7-oxabicyclo[4.1.0]hept-3-en-3-ylcarbamoyl)phenyl phosphate

In a 20-gram vial, compound 1 (300 mg, 1.15 mmol) was stirred with tetrahydrofuran (12 mL). Triethylamine (0.80 mL, 5.75 mmol) and dibenzyl chlorophosphate (3.23 mL, 1.09 mmol, 10% w:v in benzene) were added. The reaction was complete after 10 minutes as determined by LC/MS. The crude mixture was filtered and then concentrated in vacuo. The crude oil was purified by column chromatography (eluting with heptanes: ethyl acetate). The phospho-ester was obtained (450 mg, 75%, >90% pure) and the product structure was confirmed by ¹HNMR (CDCl₃): δ9.40 (br.s, 1H), 7.30 (m, 14H), 6.95 (m, 1H), 5.10 (m, 4H), 4.60 (m, 1H), 3.80 (m, 1H), 3.40 (m, 1H) ppm.

Employing the general methods described in Schemes 1-5, the following compounds may be prepared:

### Example 13

### (±)-2-(2-hydroxy-5-oxo-7-oxabicyclo[4.1.0]hept-3-en-3-ylcarbamoyl)phenyl ethylcarbamate

### Example 14

### (±)-2-(2-hydroxy-5-oxo-7-oxabicyclo[4.1.0]hept-3-en-3-ylcarbamoyl)phenyl dimethylcarbamate

### Example 15

### (±)-2-(2-hydroxy-5-oxo-7-oxabicyclo[4.1.0]hept-3-en-3-ylcarbamoyl)phenyl dihydrogen phosphate

### Example 16

### (±)-2-(2-hydroxy-5-oxo-7-oxabicyclo[4.1.0]hept-3-en-3-ylcarbamoyl)phenyl dimethyl phosphate

### Example 17

### (±)-(2-(±)-(2-hydroxy-5-oxo-7-oxabicyclo[4.1.0]hept-3-en-3-ylcarbamoyl)phenoxy)methyl acetate

The compounds of Examples 1-12 were observed to inhibit NF-κB signal transduction pathways in cells.

Two reporter cell assays were used to determine the ability of the compounds of Examples 1-12 to inhibit NF-κB driven transcription. The first assay was a 293-cell based assay with a stably integrated pNF-κB-luc reporter plasmid containing 3 NF-κB promoter elements. The second assay was a 293-cell based assay with a stably integrated pTRH1-NF-κB-dscGFP reporter containing 4 NF-κB promoter elements. Cells were treated with 0, 0.2, 1, 10, 20 and 40 μM of the compounds of Example 1-12 for 2 hours then were induced with 20 ng/ml TNF-α for 18 hours. Following the induction, luminescence or fluorescence was quantified using a Beckman-Coulter 2300 plate reader. The compounds of Example 1-12 were observed to inhibit the expression of the luciferase gene in a dose dependent manner. The compounds of Examples 1-12 also inhibited the expression of the Green fluorescent protein gene in a dose dependent manner. As a control, 0.5% DMSO treated and untreated cells were compared to verify that the compounds of Examples 1-12 had no effect on the expression of luciferase or in the readout of the assay. There was a slight decrease in the output from the assay in the DMSO treated population although it was not statistically significant. As a result of the controls, the decrease in activity in the drug treated samples was compared to the DMSO control sample.

TransAM NF-κB Family DNA Binding ELISA:

The binding activity of NF-κB heterodimer or homodimer subunits from activated nuclear extracts or purified recombinant NF-κB proteins exposed to the drug compounds was evaluated using the TransAM NF-κB Family binding ELISA (Active Motif). Approximately 3-5 μg of nuclear extracts from TNFα activated Hela or Raji cells (Active Motif) or 20 ng of purified recombinant proteins (p65 and p50 from Active Motif, p52 from Santa Cruz) were incubated for 1 hour at room temperature with 20 μL drug compounds diluted in Complete Lysis buffer without DTT. Treated samples were then transferred to 30 μL Complete Binding Buffer (with DTT) in microplate wells pre-coated with the NF-κB consensus oligonucleotide. Controls included non-specific binding (NSB) wells containing lysis buffer without any extract or recombinant protein (for background), nuclear extract or recombinant protein treated with DMSO only (for maximal binding), and wells containing the extract/protein plus 20 pmoles free wild-type NF-κB oligonucleotide as a competitor or 20 pmoles free mutant NF-κB oligonucleotide as a control to demonstrate specificity. The plate was incubated for 1 hour at room temperature with gentle shaking and then washed 3 times with 200 μL 1× Wash Buffer. NF-κB p65, p50, p52, RelB, or c-Rel subunits bound to the plate were detected with 100 μL of the primary antibody (diluted 1:1000 in 1× Antibody Buffer) specific for that subunit. The plate was incubated for 1 hour at room temperature and then washed 3 times with 200 μL 1× Wash Buffer. Next, 100 μL of a HRP conjugated goat anti-rabbit antibody (diluted 1:1,000 in 1× Antibody Buffer) was added to each well. The plate was incubated for 1 hour at room temperature and then washed 4 times with 200 μL 1× Wash Buffer. 100 μL of room temperature Developing Solution was added to each well. The reaction was allowed to develop for 2-10 minutes until a medium dark blue color developed (depending on the subunit activity in the lot of extract or lot of recombinant protein used) and then the reaction was stopped with 100 μL Stop Solution yielding a yellow color. Absorbance was recorded using a Becton-Dickinson DTX 880 Multimode Detector at 450 nm with a reference wavelength subtracted at 620 nm.

Inhibition of IL-6 and PGE2 Expressions in RAW264.7.

RAW 264.7 cells were seeded at 4×10⁴ cells per well in complete growth medium in 96 well white TC plates with clear bottoms one day prior to the assay. The next day the cells were washed once and 100 μL fresh growth media was added. Cells were pretreated with 0.5 μL from a 6 point 200× dilution series of the test compounds in DMSO for 2 hours. Following pretreatment with the drugs, the inflammatory response was induced by adding 5 μL of a 20 μg/mL solution of LPS (Sigma). The cells were incubated in the presence of the drugs and 1 μg/mL LPS for another 20-24 hours. Typically after treatment the total DMSO was 0.05% of the culture volume and the final concentrations of the compounds were approximately: 40, 20, 10, 1, 0.2 and 0 μM depending on the MW of each compound. Modified dilution series were prepared as needed to get adequate dose response curves without changing the % DMSO. Samples were run in duplicate or triplicate and included DMSO treated control wells with and without LPS stimulation. Drugs with a known activity such as Parthenolide or DHMEQ were run as experimental controls. After 20-24 hours LPS activation, the media supernatant was collected from the cells and replaced with fresh media. The supernatant samples were cleared by centrifugation at 1,000×g for 5 minutes, transferred to fresh storage plates, and stored frozen at −30° C.

After determining the appropriate supernatant dilutions experimentally, mIL-6 levels in the supernatants were quantified using Quantikine™ mouse IL-6 Immunoassay (R&D Systems) according to the manufacturer's protocol. Approximately 50 μL of the supernatants diluted in Calibrator Diluent were added to 50 μL of Assay Diluent in microplate wells pre-coated with an anti-mouse IL-6 capture antibody. Controls included a calibrated positive IL-6 control sample, non-specific binding (NSB) wells containing Calibrator Diluent but no IL-6, and a recombinant mouse IL-6 standard dilution series (10-1000 pg/mL). The plates were incubated at room temperature for 2 hours with shaking and then washed 5 times with 400 μL 1× Wash Buffer. Approximately 100 μL of an HRP-conjugated anti-mouse IL-6 antibody was added to each well to detect IL-6 captured on the plate. The plates were incubated at room temperature for 2 hours and then washed 5 times with 400 μl 1× Wash Buffer. Equal volumes of Color Reagents A and B were mixed and 100 μL of this HRP Substrate Solution was added to each well on the plate. The blue color was allowed to develop for 30 minutes and then the reaction was stopped using 100 μL of Stop Solution yielding a yellow color. Absorbance at 450 nm with a reference wavelength subtracted at 595 nm was recorded using a Becton-Dickinson DTX 880 Multimode Detector.

The concentration of mIL-6 in the unknown samples was determined from a curve-fit of the mIL-6 standard absorbance data and multiplying by the dilution factor. The maximum activity achieved in the absence of the inhibitor (DMSO+LPS treated wells) was arbitrarily given a value of 100%; likewise the minimum activity in the absence of the stimulant (no LPS) was assigned a value of 0% Inhibition of the amount of mIL-6 cytokine released in the drug treated wells was calculated relative to the maximum activation in the DMSO+LPS treated control wells (i.e., % inhibition=100−(drug+LPS treated)/(DMSO+LPS treated)). Dose response curves were used to determine the effective concentration to inhibit 50% of the mIL-6 cytokine released (IC₅₀) by means of a SigmaPlot macro which fits a sigmoidal dose-response curve to the (log 10) μM concentration versus % inhibition. In the case when compounds did not reach maximum inhibition at the concentrations tested, the curve fit was assisted with forced maximum (100%) and minimum (0%) values. This technique yields an objective value for the IC₅₀ provided that 50% inhibition was approached at the concentrations tested.

After determining the appropriate supernatant dilutions experimentally, PGE2 levels in the supernatants were quantified using Parameter™ PGE2 Immunoassay (R&D Systems) according to the manufacturer's protocol. Approximately 100 μL of the supernatants diluted in Calibrator Diluent and 50 μL of a primary monoclonal anti-PGE2 antibody were added to the microplate wells pre-coated with a goat anti-mouse Ig capture antibody. Then 50 μL of an HRP conjugated PGE2 competitor was added. Controls included non-specific binding (NSB) wells containing Calibrator Diluent but no primary antibody and a recombinant PGE2 standard dilution series (40-5000 pg/mL). The plates were incubated at room temperature for 2 hours with shaking and then washed 5 times with 400 μL 1× Wash Buffer. Equal volumes of Color Reagents A and B were mixed and 200 μL of this HRP Substrate Solution was added to each well on the plate. The blue color was allowed to develop for 30 minutes and then the reaction was stopped using 50 μL of Stop Solution yielding a yellow color. Absorbance at 450 nm with a reference at 595 nm was recorded using a Becton-Dickinson DTX 880 Multimode Detector.

The concentration of PGE2 in the unknown samples was determined from a curve-fit of the PGE2 standard absorbance data and multiplying by the dilution factor. The maximum activity achieved in the absence of the inhibitor (DMSO+LPS treated wells) was arbitrarily given a value of 100%; likewise the minimum activity in the absence of the stimulant (no LPS treated wells) was assigned a value of 0%. Inhibition of the amount of PGE2 released in the drug treated wells was calculated relative to the maximum activation in the DMSO+LPS treated control wells (i.e., % inhibition=100−(drug+LPS treated)/(DMSO+LPS treated)). Dose response curves were used to determine the effective concentration to inhibit 50% of the PGE2 released (IC₅₀) by means of a SigmaPlot macro which fits a sigmoidal dose-response curve to the (log 10) concentration versus % inhibition. In the case when compounds did not reach maximum inhibition at the concentrations tested, the curve fit was assisted with forced maximum (100%) and minimum (0%) values. This technique yields an objective value for the IC₅₀ provided that 50% inhibition was approached at the concentrations tested.

TABLE 1
Pharmacological activities of compounds in inhibition of NF-κB driven reporter gene expression,
suppression of cytokine release and inhibition of Rel protein bindings to NF-κB sites.

| | | | | | | c-Rel | RelB |
|---|---|---|---|---|---|---|---|
| | 293/NF-kB- | NF-kB/293/ | RAW 264.7 | RAW 264.7 | | binding (% | binding (% |
| | luc | GFP | IL-6 release | PGE2 release | p65 binding | inhibition at | inhibition at |
| Ex. # | EC50 (uM) | EC50 (uM) | EC50 (uM) | EC50 (uM) | IC50 (uM) | 5 uM) | 5 uM) |
| 1 | 18 | 11 | 0.5 | 1.9 | 214 | 1% | 0% |
| 2 | 11 | N/D | 1.3 | 2.7 | >376 | 0% | 2% |
| 3 | 14 | 10 | 0.72 | 1.1 | 93 | 3% | 3% |
| 4 | 10 | 17 | 0.45 | N/D | 149 | 9% | 5% |
| 5 | 15 | 6.7 | 0.44 | N/D | >430 | 9% | 5% |
| 6 | 8 | 10 | 0.35 | N/D | 67 | 6% | 3% |
| 7 | 17 | 8.4 | 1.7 | N/D | >480 | 5% | 0% |
| 8 | 10 | 14 | 1.5 | N/D | 336 | 2% | 2% |
| 9 | 15 | 16 | 1.4 | N/D | 116 | N/D | N/D |
| 10 | 24 | 13 | 6 | N/D | 59 | N/D | N/D |
| 11 | 3.3 | 4.3 | 0.87 | N/D | 7.3 | N/D | N/D |
| 12 | >26 | >26 | >26 | N/D | >320 | N/D | N/D |

Without further description, it is believed that one of ordinary skill in the art can, using the preceding description and the following illustrative examples, make and utilize the compounds of the present invention and practice the claimed methods. While the invention has been described and illustrated herein by references to various specific materials, procedures and examples, it is understood that the invention is not restricted to the particular combinations of material and procedures selected for that purpose. Numerous variations of such details can be implied as will be appreciated by those skilled in the art. All patents, patent applications and other references cited throughout this application are herein incorporated by reference in their entirety.

### REFERENCES

Aggarwal B B, Shishodia S, Sandur S K, Pandey M K, Sethi G (2006) Inflammation and cancer: how hot is the link? Biochem Pharmacol 72(11): 1605-1621.
Arcaroli J, Silva E, Maloney J P, He Q, Svetkauskaite D, Murphy J R, Abraham E (2006) Variant IRAK-1 haplotype is associated with increased nuclear factor-kappaB activation and worse outcomes in sepsis. Am J Respir Crit Care Med 173(12): 1335-1341.
Argyropoulos C, Mouzaki A (2006) Immunosuppressive drugs in HIV disease. Curr Top Med Chem 6(16): 1769-1789.
Ariga A, Namekawa J-i, Matsumoto N, Inoue J-i, Umezawa K (2002) Inhibition of tumor necrosis factor0alpha-induced nuclear translocation and activation of NF-kappaB by dehydroxymethylepoxyquinomicin. J. Biol. Chem. 277(27): 24625-24630.
Atreya I, Atreya R, Neurath M F (2008) NF-kappaB in inflamatory bowel disease. J Intern Med 263(6): 591-596.
Baba M (2006) Recent status of HIV-1 gene expression inhibitors. Antiviral Res 71(2-3): 301-3 Barnes P J (1997) Nuclear factor-kappa B. Int J Biochem Cell Biol 29(6): 867-870.
Bosisio D, Marazzi I, Agresti A, Shimizu N, Bianchi M E, Natoli G (2006) A hyper-dynamic equilibrium between promoter-bound and nucleoplasmic dimers controls NF-kappaB-dependent gene activity. Embo J 25(4): 798-810.
Bouma G, Strober W (2003) The immunological and genetic basis of inflammatory bowel disease. Nat Rev Immunol 3(7): 521-533.
Burstein E, Duckett C S (2003) Dying for NF-kappaB? Control of cell death by transcriptional regulation of the apoptotic machinery. Curr Opin Cell Biol 15(6): 732-737.
Calzado M A, Bacher S, Schmitz M L (2007) NF-kappaB inhibitors for the treatment of inflammatory diseases and cancer. Curr Med Chem 14(3): 367-376.
Chaicharoenpong C, Kato K, Umezawa K (2002) Synthesis and structure-activity relationship of dehydroxymethylepoxyquinomicin analogues as inhibitors of NF-kappaB functions. Bioorg Med Chem 10(12): 3933-3999.
Flory E, Kunz M, Scheller C, Jassoy C, Stauber R, Rapp U R, Ludwig S (2000) Influenza virus-induced NF-kappaB-dependent gene expression is mediated by overexpression of viral proteins and involves oxidative radicals and activation of IkappaB kinase. J Biol Chem 275(12): 8307-8314.
Giri R K, Rajagopal V, Shahi S, Zlokovic B V, Kalra V K (2005) Mechanism of amyloid peptide induced CCR5 expression in monocytes and its inhibition by siRNA for Egr-1. Am J Physiol Cell Physiol 289(2): C264-276.
Goldring S R, Jasty M, Roelke M S, Rourke C M, Bringhurst F R, Harris W H (1986) Formation of a synovial-like membrane at the bone-cement interface. Its role in bone resorption and implant loosening after total hip replacement. Arthritis Rheum 29(7): 836-842.
Greten F R, Karin M (2004) The IKK/NF-kappaB activation pathway-a target for prevention and treatment of cancer. Cancer Lett 206(2): 193-199.
Hacker H, Karin M (2006) Regulation and function of IKK and IKK-related kinases. Sci STKE 2006(357): rel3.
Harris W H (1995) The problem is osteolysis. Clin Orthop Relat Res (311): 46-53.
Hayden M S, Ghosh S (2004) Signaling to NF-kappaB. Genes Dev 18(18): 2195-2224.
Hayden M S, West A P, Ghosh S (2006a) NF-kappaB and the immune response. Oncogene 25(51): 6758-6780.
Hayden M S, West A P, Ghosh S (2006b) SnapShot: NF-kappaB Signaling Pathways. Cell 127(6): 1286-1287.
Helbig G, Christopherson K W, 2nd, Bhat-Nakshatri P, Kumar S, Kishimoto H, Miller K D, Broxmeyer H E, Nakshatri H (2003) NF-kappaB promotes breast cancer cell migration and metastasis by inducing the expression of the chemokine receptor CXCR4. J Biol Chem 278(24): 21631-21638.
Iordanskiy S, lordanskaya, T, Quivy V, Van Lint C, Bukrinsky M (2002) B-oligomer of pertussis toxin inhibits HIV-1 LTR-driven transcription through suppression of NF-kappaB p65 subunit activity. Virology 302(1): 195-206.
Israel A (2000) The IKK complex: an integrator of all signals that activate NF-kappaB? Trends Cell Biol 10(4): 129-133.
Jung M, Zhang Y, Lee S, Dritschilo A (1995) Correction of radiation sensitivity in ataxia telangiectasia cells by a truncated I kappa B-alpha. Science 268(5217): 1619-1621.
Kalergis A M, Iruretagoyena M I, Barrientos M J, Gonzalez P A, Herrada A A, Leiva E D, Gutierrez M A, Riedel C A, Bueno S M, Jacobelli S H (2008) Modulation of nuclear factor-kappaB activity can influence the susceptibility to systemic lupus erythematosus. Immunology.
Karin M (1999) How NF-kappaB is activated: the role of the IkappaB kinase (IKK) complex. Oncogene 18(49): 6867-6874.
Karin M (2006) Nuclear factor-kappaB in cancer development and progression. Nature 441(7092): 431-436.
Ketas T J, Kuhmann S E, Palmer A, Zurita J, He W, Ahuja S K, Klasse P J, Moore J P (2007) Cell surface expression of CCR5 and other host factors influence the inhibition of HIV-1 infection of human lymphocytes by CCR5 ligands. Virology 364(2):281-90.
Kurylowicz A, Nauman J (2008) The role of nuclear factor-kappaB in the development of autoimmune diseases: a link between genes and environment. Acta Biochim Pol 55(4): 629-647.
Lawrence R C, Helmick C G, Arnett F C, Deyo R A, Felson D T, Giannini E H, Heyse S P, Hirsch R, Hochberg M C, Hunder G G, Liang M H, Pillemer S R, Steen V D, Wolfe F (1998) Estimates of the prevalence of arthritis and selected musculoskeletal disorders in the United States. Arthritis Rheum 41(5): 778-799.
Liao F, Andalibi A, Qiao J H, Allayee H, Fogelman A M, Lusis A J (1994) Genetic evidence for a common pathway mediating oxidative stress, inflammatory gene induction, and aortic fatty streak formation in mice. J Clin Invest 94(2): 877-884.
Liu R, Zhao X, Gurney T A, Landau N R (1998) Functional analysis of the proximal CCR5 promoter. AIDS Res Hum Retroviruses 14(17): 1509-1519,
Maruyama I, Shigeta K, Miyahara H, Nakajima T, Shin H, Ide S, Kitajima I (1997) Thrombin activates NF-kappa B through thrombin receptor and results in proliferation of vascular smooth muscle cells: role of thrombin in atherosclerosis and restenosis. Ann N Y Acad Sci 811: 429-436.
Mukerjee R, Sawaya B E, Khalili K, Amini S (2006) Association of p65 and C/EBPbeta with HIV-1 LTR modulates transcription of the viral promoter. J Cell Biochem 100(5):1210-6.
Natoli G, Saccani S, Bosisio D, Marazzi I (2005) Interactions of NF-kappaB with chromatin: the art of being at the right place at the right time. Nat Immunol 6(5): 439-445.
Neurath M F, Fuss I, Schurmann G, Pettersson S, Arnold K, Muller-Lobeck H, Strober W, Herfarth C, Buschenfelde K H (1998) Cytokine gene transcription by NF-kappa B family members in patients with inflammatory bowel disease. Ann N Y Acad Sci 859: 149-159.
Niu J, Azfer A, Kolattukudy P E (2008) Protection against lipopolysaccharide-induced myocardial dysfunction in mice by cardiac-specific expression of soluble Fas. J Mol Cell Cardiol 44(1): 160-169.
Pahl H L, Baeuerle P A (1995) Expression of influenza virus hemagglutinin activates transcription factor NF-kappa B. J Virol 69(3): 1480-1484.
Palmieri C, Trimboli F, Puca A, Fiume G, Scala G, Quinto I (2004) Inhibition of HIV-1 replication in primary human monocytes by the IkappaB-alphaS32/36A repressor of NF-kappaB. Retrovirology 1(1): 45.
Pihlstrom B L, Michalowicz B S, Johnson N W (2005) Periodontal diseases. Lancet 366(9499): 1809-1820.
Platt E J, Wehrly K, Kuhmann S E, Chesebro B, Kabat D (1998) Effects of CCR5 and CD4 cell surface concentrations on infections by macrophagetropic isolates of human immunodeficiency virus type 1. J Virol 72(4): 2855-2864.
Reeves J D, Gallo S A, Ahmad N, Miamidian J L, Harvey P E, Sharron M, Pohlmann S, Sfakianos J N, Derdeyn C A, Blumenthal R, Hunter E, Doms R W (2002) Sensitivity of HIV-1 to entry inhibitors correlates with envelope/coreceptor affinity, receptor density, and fusion kinetics. Proc Nail Acad Sci U S A 99(25): 16249-16254.
Rizzi C, Alfano M, Bugatti A, Camozzi M, Poli G, Rusnati M (2004) Inhibition of intra- and extra-cellular Tat function and HIV expression by pertussis toxin B-oligomer. Eur J Immunol 34(2): 530-536.
Saccani S, Marazzi I, Beg A A, Natoli G (2004) Degradation of promoter-bound p65/RelA is essential for the prompt termination of the nuclear factor kappaB response. J Exp Med 200(1): 107-113.
Scheidereit C (2006) IkappaB kinase complexes: gateways to NF-kappaB activation and transcription. Oncogene 25(51): 6685-6705.
Sui Z, Sniderhan L F, Fan S, Kazmierczak K, Reisinger E, Kovacs A D, Potash M J, Dewhurst S, Gelbard H A, Maggirwar S B (2006) Human immunodeficiency virus-encoded Tat activates glycogen synthase kinase-3beta to antagonize nuclear factor-kappaB survival pathway in neurons. Eur J Neurosci 23(10): 2623-2634.
Suzuki, Y, Sugiyama, C, Ohno, O and Umezawa, K (2004) Preparation and biological activities of optically active dehydroxymethylepoxyquinomicin, a novel NF-κB inhibitor, Tetrahedron, 60; 7061-7066.
Takeuich T, Umezawa K, To-E S, Matsumoto N, Sawa T, Yoshioka T, Agata N, Hirano S-i, Isshiki K (2003) Salicylamide derivatives. U.S. Pat. No. 6,566,394 B1.
Tergaonkar V (2006) NFkappaB pathway: a good signaling paradigm and therapeutic target. Int J Biochem Cell Biol 38(10): 1647-1653.
Tilg H, Moschen A R, Kaser A, Pines A, Dotan I (2008) Gut, inflammation and osteoporosis: basic and clinical concepts. Gut 57(5): 684-694.
Suzuki Y, Sugiyama C, Ohno O, Umezawa K (2004) Preparation and biological activities of optically active dehydroxymethylepoxyquinomicin, a novel NF-kB inhibitor. Tetrahedron 60:7061-7066.
Umezawa K (2006) Inhibition of tumor growth by NF-kappaB inhibitors. Cancer Sci 97(10):990-995.
Williams S A, Chen L F, Kwon H, Ruiz-Jarabo C M, Verdin E, Greene W C (2006) NF-kappaB p50 promotes HIV latency through HDAC recruitment and repression of transcriptional initiation. EMBO J 25(1): 139-149,
Williams S A, Kwon H, Chen L F, Greene W C (2007) Sustained Induction of NF-{kappa} B Is Required for Efficient Expression of Latent HIV-1. J Virol 81(11):6043-56.
Wirtz S, Neurath M F (2007) Mouse models of inflammatory bowel disease. Adv Drug Deliv Rev 59(11): 1073-1083.
Yamamoto M, Horie R, Takeiri M, Kozawa I, Umezawa K (2008) Inactivation of NF-kappaB components by covalent binding of (−)-dehydroxymethylepoxyquinomicin to specific cysteine residues. J. Med Chem 51(8):5780-5788.

1. A compound of formula (I) 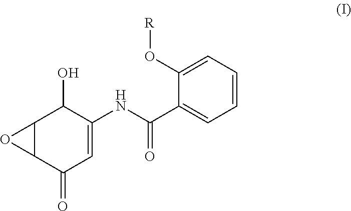
or a pharmaceutically acceptable salt thereof,
wherein
R is COR¹, CONHR¹, CONR¹R¹, COOR¹, CH₂OCOR¹, P(O)(OH)₂, P(O)(O(C1-C6)alkyl)₂, P(O)(O(C1-C6)alkylphenyl)₂, P(O)(OCH₂OCO(C1-C6)alkyl)₂, P(O)(OH)(OCH₂OCO(C1-C6)alkyl), P(O)(OH)(O(C1-C6)alkyl), P(O)(OH)((C1-C6)alkyl), glycosyl or a salt thereof, wherein
each R¹ is independently (C1-C6)alkyl, trifluoromethyl, cycloalkyl, heterocycloalkyl, aryl, alkylaryl, heteroaryl or alkylheteroaryl, wherein the aryl or heteroaryl ring is substituted with 0 to 4 groups selected from the group consisting of fluorine, chlorine, bromine, cyano, hydroxyl, amino, trifluoromethyl, (C1-C4)alkyl, (C1-C4)alkoxy, pyridinyl, pyrimidinyl or benzyl optionally substituted with fluorine, chlorine, bromine, hydroxyl, trifluoromethyl, (C1-C4)alkyl or (C1-C4)alkoxy.

2. A compound of formula (II) 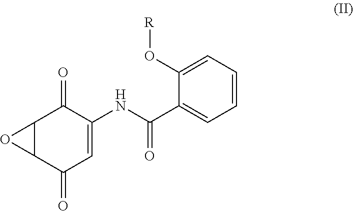
or a pharmaceutically acceptable salt thereof,
wherein
R is COR¹, CONHR¹, CONR¹R¹, COOR¹, CH₂OCOR¹, P(O)(OH)₂, P(O)(O(C1-C6)alkyl)₂, P(O)(O(C1-C6)alkylphenyl)₂, P(O)(OCH₂OCO(C1-C6)alkyl)₂, P(O)(OH)(OCH₂OCO(C1-C6)alkyl), P(O)(OH)(O(C1-C6)alkyl), P(O)(OH)((C1-C6)alkyl), glycosyl or a salt thereof, wherein
each R¹ is independently (C1-C6)alkyl, trifluoromethyl, cycloalkyl, heterocycloalkyl, aryl, alkylaryl, heteroaryl or alkylheteroaryl, wherein the aryl or heteroaryl ring is substituted with 0 to 4 groups selected from the group consisting of fluorine, chlorine, bromine, cyano, hydroxyl, amino, trifluoromethyl, (C1-C4)alkyl, (C1-C4)alkoxy, pyridinyl, pyrimidinyl or benzyl optionally substituted with fluorine, chlorine, bromine, hydroxyl, trifluoromethyl, (C1-C4)alkyl or (C1-C4)alkoxy.

3. A compound of formula (III) 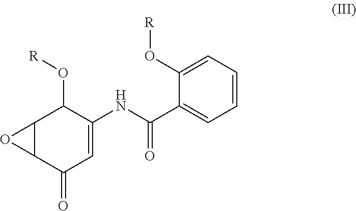
or a pharmaceutically acceptable salt thereof,
wherein
each R is independently COR¹, CONHR¹, CONR¹R¹, COOR¹, CH₂OCOR¹, P(O)(OH)₂, P(O)(O(C1-C6)alkyl)₂, P(O)(O(C1-C6)alkylphenyl)₂, P(O)(OCH₂OCO(C1-C6)alkyl)₂, P(O)(OH)(OCH₂OCO(C1-C6)alkyl), P(O)(OH)(O(C1-C6)alkyl), P(O)(OH)((C1-C6)alkyl), glycosyl or a salt thereof, wherein
each R¹ is independently (C1-C6)alkyl, trifluoromethyl, cycloalkyl, heterocycloalkyl, aryl, alkylaryl, heteroaryl or alkylheteroaryl, wherein the aryl or heteroaryl ring is substituted with 0 to 4 groups selected from the group consisting of fluorine, chlorine, bromine, cyano, hydroxyl, amino, trifluoromethyl, (C1-C4)alkyl, (C1-C4)alkoxy, pyridinyl, pyrimidinyl or benzyl optionally substituted with fluorine, chlorine, bromine, hydroxyl, trifluoromethyl, (C1-C4)alkyl or (C1-C4)alkoxy.

4. A compound of formula (IV) 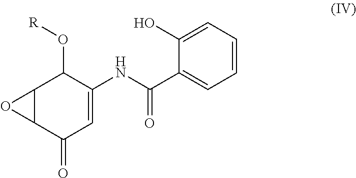
or a pharmaceutically acceptable salt thereof,
wherein
R is COR¹, CONHR¹, CONR¹R¹, COOR¹, CH₂OCOR¹, P(O)(OH)₂, P(O)(O(C1-C6)alkyl)₂, P(O)(O(C1-C6)alkylphenyl)₂, P(O)(OCH₂OCO(C1-C6)alkyl)₂, P(O)(OH)(OCH₂OCO(C1-C6)alkyl), P(O)(OH)(O(C1-C6)alkyl), P(O)(OH)((C1-C6)alkyl), glycosyl or a salt thereof, wherein
each R¹ is independently (C1-C6)alkyl, trifluoromethyl, cycloalkyl, heterocycloalkyl, aryl, alkylaryl, heteroaryl or alkylheteroaryl, wherein the aryl or heteroaryl ring is substituted with 0 to 4 groups selected from the group consisting of fluorine, chlorine, bromine, cyano, hydroxyl, amino, trifluoromethyl, (C1-C4)alkyl, (C1-C4)alkoxy, pyridinyl, pyrimidinyl or benzyl optionally substituted with fluorine, chlorine, bromine, hydroxyl, trifluoromethyl, (C1-C4)alkyl or (C1-C4)alkoxy.

5. A pharmaceutical composition comprising a compound according to claim 1 or a pharmaceutically acceptable salt thereof in combination with a pharmaceutically effective diluent or carrier.

6. A method of treating a disease in a mammal associated with inhibition of activation of NF-κB, comprising administering to a mammal in need thereof a therapeutically effective amount of a compound of formula (I) or a pharmaceutically acceptable salt thereof according to claim 1.

7. A method of treating a disease in a mammal associated with inhibition of activation of NF-κB, comprising administering to a mammal in need thereof a therapeutically effective amount of a compound of formula (II) or a pharmaceutically acceptable salt thereof according to claim 2.

8. A method of treating a disease in a mammal associated with inhibition of activation of NF-κB, comprising administering to a mammal in need thereof a therapeutically effective amount of a compound of formula (III) or a pharmaceutically acceptable salt thereof according to claim 3.

9. A method of treating a disease in a mammal associated with inhibition of activation of NF-κB, comprising administering to a mammal in need thereof a therapeutically effective amount of a compound of formula (IV) or a pharmaceutically acceptable salt thereof according to claim 4.

10. The method of claim 6, wherein the disease is selected from the group consisting of cancer, inflammation, auto-immune diseases, diabetes, infection, cardiovascular disease and ischemia-reperfusion injuries.

11. The method of claim 7, wherein the disease is selected from the group consisting of cancer, inflammation, auto-immune diseases, diabetes, infection, cardiovascular disease and ischemia-reperfusion injuries.

12. The method of claim 8, wherein the disease is selected from the group consisting of cancer, inflammation, auto-immune diseases, diabetes, infection, cardiovascular disease and ischemia-reperfusion injuries.

13. The method of claim 9, wherein the disease is selected from the group consisting of cancer, inflammation, auto-immune diseases, diabetes, infection, cardiovascular disease and ischemia-reperfusion injuries.

14. A pharmaceutical composition comprising a compound according to claim 2 or a pharmaceutically acceptable salt thereof in combination with a pharmaceutically effective diluent or carrier.

15. A pharmaceutical composition comprising a compound according to claim 3 or a pharmaceutically acceptable salt thereof in combination with a pharmaceutically effective diluent or carrier.

16. A pharmaceutical composition comprising a compound according to claim 4 or a pharmaceutically acceptable salt thereof in combination with a pharmaceutically effective diluent or carrier.

17. The compound according to claim 1, which is:
(±)-2-(2-hydroxy-5-oxo-7-oxabicyclo[4.1.0]hept-3-en-3-ylcarbamoyl)phenyl 3-methylbutanoate;
(±)-2-(2-hydroxy-5-oxo-7-oxabicyclo[4.1.0]hept-3-en-3-ylcarbamoyl)phenyl 2-cyclohexylacetate;
(±)-2-(2-hydroxy-5-oxo-7-oxabicyclo[4.1.0]hept-3-en-3-ylcarbamoyl)phenyl 2-methylpentanoate;
(±)-2-(2-hydroxy-5-oxo-7-oxabicyclo[4.1.0]hept-3-en-3-ylcarbamoyl)phenyl 2-ethylhexanoate;
(±)-2-(2-hydroxy-5-oxo-7-oxabicyclo[4.1.0]hept-3-en-3-ylcarbamoyl)phenyl 3,3-dimethylbutanoate;
(±)-2-(2-hydroxy-5-oxo-7-oxabicyclo[4.1.0]hept-3-en-3-ylcarbamoyl)phenyl isopropyl carbonate;
(±)-diethyl 2-(2-hydroxy-5-oxo-7-oxabicyclo[4.1.0]hept-3-en-3-ylcarbamoyl)phenyl phosphate;
(±)-dibenzyl 2-(2-hydroxy-5-oxo-7-oxabicyclo[4.1.0]hept-3-en-3-ylcarbamoyl)phenyl phosphate;
(±)-2-(2-hydroxy-5-oxo-7-oxabicyclo[4.1.0]hept-3-en-3-ylcarbamoyl)phenyl ethylcarbamate;
(±)-2-(2-hydroxy-5-oxo-7-oxabicyclo[4.1.0]hept-3-en-3-ylcarbamoyl)phenyl dimethylcarbamate;
(±)-2-(2-hydroxy-5-oxo-7-oxabicyclo[4.1.0]hept-3-en-3-ylcarbamoyl)phenyl dihydrogen phosphate;
(±)-2-(2-hydroxy-5-oxo-7-oxabicyclo[4.1.0]hept-3-en-3-ylcarbamoyl)phenyl dimethyl phosphate;
(±)-(2-(±)-(2-hydroxy-5-oxo-7-oxabicyclo[4.1.0]hept-3-en-3-ylcarbamoyl)phenoxy)methyl acetate;
or a pharmaceutically acceptable salt thereof.

18. The compound according to claim 3, which is:
(±)-3-(2-isopropoxycarbonyloxy-benzoylamino)-5-oxo-7-oxa-bicyclo[4.1.0]hept-3-en-2-yl isopropyl ester;
(±)-2-(2-(3,3-dimethylbutanoyloxy)-5-oxo-7-oxabicyclo[4.1.0]hept-3-en-3-ylcarbamoyl)phenyl 3,3-dimethylbutanoate;
or a pharmaceutically acceptable salt thereof.

19. The compound according to claim 4, which is (±)-3-(2-hydroxybenzamido)-5-oxo-7-oxabicyclo[4.1.0]hept-3-en-2-yl phenylcarbamate, 3-(2-hydroxybenzamido)-5-oxo-7-oxabicyclo[4.1.0]hept-3-en-2-yl isopropyl carbonate or a pharmaceutically acceptable salt thereof.
